(19) United States
(12) Patent Application Publication
(10) Pub. No.: US 2025/0262262 A1
(43) Pub. Date: Aug. 21, 2025

(54) a-ACID AND/OR B-ACID BASED COPOSITIONS FROM HUMULUS FOR USE IN A HUMAN AND/OR VETERINARY APPLICATION

(71) Applicant: Orotech NV, Temse (BE)

(72) Inventors: Brenda Vermeulen, Kruibeke (BE); Jean-Paul Remon, Melle (BE)

(21) Appl. No.: 18/859,415

(22) PCT Filed: Apr. 28, 2023
(86) PCT No.: PCT/EP2023/061285

§ 371 Date: Oct. 23, 2024

(30) Foreign Application Priority Data
Apr. 29, 2022 (EP) 22170711.0

(51) Int. Cl. A61K 36/185; A61K 8/02; A61K 8/35; A61K 8/73; A61K 8/81; A61K 8/9789; A61K 9/00; A61K 9/14; A61K 9/20; A61K 31/122; A61P 33/02

(52) U.S. Cl. CPC A61K 36/3486; A61K 8/0204; A61K 8/35; A61K 8/731; A61K 8/8182; A61K 8/9789; A61K 9/0053; A61K 9/146; A61K 9/2013; A61K 9/2027; A61K 9/2054; A61K 31/122; A61P 33/02; A61K 2800/805

(57) ABSTRACT

The present invention relates to an α-acid and/or a β-acid based composition, yielded in a hot-melt extrusion (HME) process from Humulus, in particular for use in human and/or veterinary application. Furthermore, the invention also relates to a topical formulation comprising said composition, an oral formulation comprising said composition, a use of said composition and/or oral formulation, a method for preparation of said composition, and use of hydroxypropylmethylcellulose and/or vinylpyrrolidine-vinyl acetate copolymer in a hot-melt extrusion process of an α-acid and/or a β-acid from Humulus and/or an α-acid and/or a β-acid as a semi-synthetic form.

### FIELD OF THE INVENTION

The present invention relates to an α-acid and/or a β-acid based composition, yielded in a hot-melt extrusion (HME) process from Humulus, in particular for use in human and/or veterinary application. Furthermore, the invention also relates to a topical formulation comprising said composition, an oral formulation comprising said composition, a use of said composition and/or oral formulation, a method for preparation of said composition, and use of hydroxypropylmethylcellulose and/or vinylpyrrolidone-vinyl acetate copolymer in a hot-melt extrusion process of an α-acid and/or a β-acid from Humulus, such as in the form of a Humulus extract, in semi-synthetic form or in a salt form thereto, either or not fixed on a carrier.

### BACKGROUND TO THE INVENTION

α-acids and/or a β-acids are acids which may be derived from Humulus, wherein Humulus may also be referred to as hop. Said acids are renowned to exhibit a broad range of bioactivities, but are specifically renowned for their suppression of inflammatory responses and their capacity to attenuate neural hyperactivation in the hippocampus, immunomodulatory activity, metabolic syndromes, against obesity and fatty liver disease, irritable bowl syndrome, improved sleep induction, reduction of anxiety levels, reduction of stress levels, antioxidant activity, and reduction of depression symptoms. α-acids and/or a β-acids may also be used in the prevention of coccidiosis, and may be added to a feed supplement.

Due to their bioactivity, they are of course also an interesting component in topical formulations. However, α-acids and/or a β-acids-containing compositions typically have a low bioavailability.

Therefore, until now, it has been very difficult to formulate α-acids and/or a β-acids having good bioavailability of its components.

Thus, there is a need to develop new formulations that contain high concentrations of bioavailable α-acids and/or a β-acids.

It was surprisingly found that hot-melt extrusion of an α-acid and/or a β-acid in the presence of a water-soluble nutritionally and/or cosmetically and/or pharmaceutically and/or therapeutically and/or nutraceutical acceptable thermoplastic polymer such as for example hydroxypropylmethylcellulose (HPMC) or a vinylpyrrolidone-vinyl acetate copolymer, followed by milling resulted in a powder suitable for use in water-containing formulations and/or use in solid formulations. It was surprisingly found that the claimed compositions could easily be hot-melt extruded without having the α-acids and/or a β-acids losing their activity. Moreover, after supplementation of water with these extrudates, the extrudate could easily be dissolved in water, whereafter the α-acids and/or a β-acids remained dissolved in the water, without sedimentation. Accordingly, these extrudates are highly suitable for drinking water supplementation or formulation in aqueous compositions.

WO2009142736 discloses a process for spray-drying hop extracts with maltodextrin and modified starch. As evident from example 4, the process disclosed therein results in products having significantly different characteristics compared to the products obtained using the process of the invention.

WO2018189311 discloses a process for hot-melt extrusion of xanthohumol extracts. These extracts at most contain only marginal amounts of alpha- and/or beta-acids in contrast to the compositions of the present invention.

### SUMMARY OF THE INVENTION

Accordingly, in a first aspect, the present invention provides a composition comprising an α-acid and/or a β-acid from Humulus and/or an α-acid and/or a β-acid as a semi-synthetic form or in a salt form thereof and a hydroxypropylmethylcellulose and/or a vinylpyrrolidone-vinyl acetate copolymer, wherein said composition is in the form of an hot-melt extrudate. In particular, the α-acid is present at a concentration of at least 10 wt. % of the dry weight of the composition, and/or the β-acid is present at a concentration of at least 10 wt. % of the dry weight of the composition of the dry weight of the composition.

It is noted that throughout the application semi-synthetic form refers to an α-acid and/or a β-acid, for example wherein the α-acid and/or the β-acid originates from Humulus, which has been modified such as to obtain an iso-reduced form thereof.

Furthermore, it is noted that throughout this application α-acid and/or β-acid (or a derivate thereof) may refer to the free acid of α-acid and/or β-acid, salt form of α-acid and/or β-acid (e.g. potassium and sodium salts of α-acid and/or β-acid), and/or salt form of α-acid and/or β-acid bound to a (solid) carrier such as a silica carrier.

In a particular embodiment, the present invention provides a composition as defined herein, further comprising a plasticizer. Preferably, the plasticizer is polyethylene glycol (PEG) and/or polyglyceryl-3 polyricinoleate.

In a further particular embodiment, the present invention provides a composition as defined herein, wherein the α-acid is present in the range of 0.5 wt. % to 50 wt. % of the dry weight of the composition, preferably in the range of 0.5 wt. % to 30 wt. % of the dry weight of the composition. In particular, the α-acid may be present in the range of 10 wt. % to 50 wt. % of the dry weight of the composition, preferably in the range of 10 wt. % to 30 wt. % of the dry weight of the composition.

In yet a further particular embodiment, the present invention provides a composition as defined herein, wherein the β-acid is present in the range of 0.5 wt. % to 50 wt. % of the dry weight of the composition, preferably in the range of 0.5 wt. % to 30 wt. % of the dry weight of the composition. In particular, the β-acid may be present in the range of 10 wt. % to 50 wt. % of the dry weight of the composition, preferably in the range of 10 wt. % to 30 wt. % of the dry weight of the composition.

In yet a further particular embodiment, the present invention provides a composition as defined herein, wherein the α-acid is an iso-α-acid. Preferably, wherein the iso-α-acid is a tetra-hydro-iso-α-acid and/or hexa-hydro-iso-α-acid.

In yet a further particular embodiment, the present invention provides a composition as defined herein, wherein the α-acid is one or more selected from the group of humulone, adhumulone, cohumulone, posthumulone, and prehumulone, preferably the α-acid is cis-iso-humulone and/or trans-iso-humulone.

In yet a further particular embodiment, the present invention provides a composition as defined herein, wherein the β-acid is lupulone.

In yet a further particular embodiment, the present invention provides a composition as defined herein, further comprising xanthohumol, preferably xanthohumol extrudate.

In yet a further particular embodiment, the present invention provides a composition as defined herein, further comprising one or more additive selected from the group of microcrystalline cellulose, lactose, dextrose, sucrose, sorbitol, mannitol, maltodextrin, di-basic calcium phosphate, cellulose, pregelatinized starch, croscarmellose sodium, crospovidone, sodium starch glycolate, calcium carbonate, magnesium carbonate, alginic acid, hydroxypropylmethylcellulose, hydroxypropylcellulose, soy polysaccharide, xylan, xanthan gum, aluminium silicate, magnesium silicate, colloidal silica, aluminium stearate, calcium stearate, magnesium stearate, zinc stearate, magnesium lauryl sulphate, sodium lauryl sulphate, sodium stearyl fumarate, glyceryl tristearate, glyceryl monostearate, glyceryl behenate, palmitic acid, stearic acid, palmitoyl alcohol, stearyl alcohol, hydrogenated vegetable oil, hydrogenated castor oil, mineral oil, fumaric acid, starch, talc, sodium benzoate, calcium silicate, magnesium silicate, magnesium oxide, colloidal silicon dioxide, cellulose, starch, talc.

In yet a further particular embodiment, the present invention provides a composition as defined herein, wherein the α-acid and/or the β-acid originate from Humulus lupulus and/or are semi-synthetic α-acid and/or semi-synthetic β-acid.

In yet a further particular embodiment, the present invention provides a composition as defined herein, wherein the extrudate is in the form of an extrudate as such, a powder, a nanoparticulate form or a microparticulate form.

In a further aspect, the present invention provides a topical formulation comprising the composition according to the invention. Preferably, the topical formulation may be a topical cosmetic formulation and/or a topical pharmaceutical formulation and/or a topical therapeutic formulation and/or topical nutraceutical formulation.

It is noted that throughout this application topical formulation may be changed by topical cosmetic formulation and/or topical pharmaceutical formulation and/or topical nutraceutical formulation.

In a particular embodiment, the present invention provides a topical formulation as defined herein, wherein said formulation is in the form of a gel, cream, foam, paste, lotion, milk, emulsion, solution, suspension, ointment, lipstick, shower gel, bath gel, shampoo, sunscreen, after sun preparation, spray, moisturizer, anti-dandruff formulation, antiperspirant or deodorant composition.

In a further particular embodiment, the present invention provides a topical formulation as defined herein, wherein said formulation further comprises one or more components selected from the list comprising: hydration agents, vitamins, antioxidants, peptides, plant extracts, anti-slimming components, anti-ageing components, anti-acne components, anti-inflammatory components, stimulants, sleep inducing agents, metabolic agents and cognitive improving agents.

In a further particular embodiment, the present invention provides a topical formulation as defined herein, wherein said formulation further comprises one or more components selected from the list comprising: hydration agents, vitamins, antioxidants, peptides, plant extracts, anti-slimming components, anti-ageing components, anti-acne components, anti-inflammatory components.

In a further aspect, the present invention provides an oral formulation comprising the composition according to the invention.

In a particular embodiment, the present invention provides an oral formulation as defined herein, which is in the form of a pharmaceutical formulation, a therapeutical formulation, a nutraceutical formulation, a food supplement, or a feed supplement.

In a further particular embodiment, the present invention provides an oral formulation as defined herein, wherein said formulation is in the form of an extrudate, a powder, uncoated or coated capsule, uncoated or coated tablet, pill, lozenge, sachet, cachet, elixir, suspension, emulsion, solution, syrup, uncoated or coated soft and hard gelatin capsules, granule or uncoated or coated pellet.

In a further particular embodiment, the present invention provides an oral formulation as defined herein, wherein said formulation further comprises one or more components selected from the list comprising: hydration agents, vitamins, antioxidants, peptides, plant extracts, anti-slimming components, anti-ageing components, anti-acne components, anti-inflammatory components.

In a further aspect, the present invention provides the composition according to the invention and/or the oral formulation according to the invention for use in human and/or veterinary application.

In a further aspect, the present invention provides a method for the preparation of a composition according to the invention, comprising the steps of:
mixing an α-acid and/or a β-acid from hop and/or a semi-synthetic α-acid and/or a semi-synthetic β-acid with hydroxypropylmethylcellulose and/or vinylpyrrolidone-vinyl acetate copolymer;
hot-melt extruding said mixture; and
optionally milling said hot-melt extruded mixture.

In particular, in such process, the α-acid is present at a concentration of at least 10 wt. % of the dry weight of the composition, and/or the β-acid is present at a concentration of at least 10 wt. % of the dry weight of the composition of the dry weight of the composition

In a particular embodiment, the present invention provides a method as defined herein, wherein the α-acid is an iso-α-acid. Preferably, wherein the iso-α-acid is a tetra-hydro-iso-α-acid and/or hexa-hydro-iso-α-acid.

In a further particular embodiment, the present invention provides a method as defined herein, wherein the step of mixing further comprises mixing a plasticizer and/or xanthohumol with the α-acid and/or the β-acid and/or the semi-synthetic α-acid and/or the semi-synthetic β-acid and hydroxypropylmethylcellulose and/or vinylpyrrolidone-vinyl acetate copolymer.

In a further aspect, the present invention provides a use of hydroxypropylmethylcellulose and/or vinylpyrrolidine-vinyl acetate copolymer in a hot-melt extrusion process of an α-acid and/or a β-acid from Humulus and/or an α-acid and/or a β-acid as a semi-synthetic form, preferably in a hot-melt extrusion process of an iso-α-acid from Humulus and/or an iso-α-acid as a semi-synthetic form.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIGS. 1A & B: Comparison of the solubility of products obtained using a prior art process as disclosed in WO2009142736 (left panels) versus products obtained using the process of the present invention (right panels)

### DETAILED DESCRIPTION

Accordingly, as already defined herein above, the present invention provides a composition comprising an α-acid and/or a β-acid from Humulus and/or an α-acid and/or a β-acid as a semi-synthetic form or in salt form thereof, and a hydroxypropylmethylcellulose and/or a vinylpyrrolidone-vinyl acetate copolymer, wherein said composition is in the form of an hot-melt extrudate.

It is noted that α-acid and β-acid may respectively be represented by any of the following formulas:

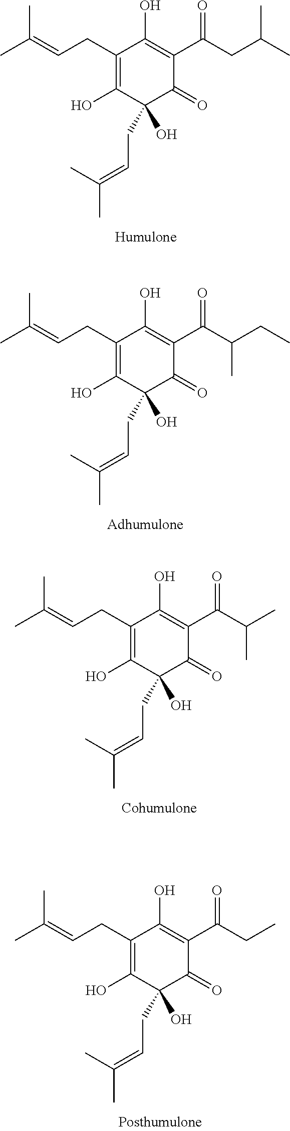

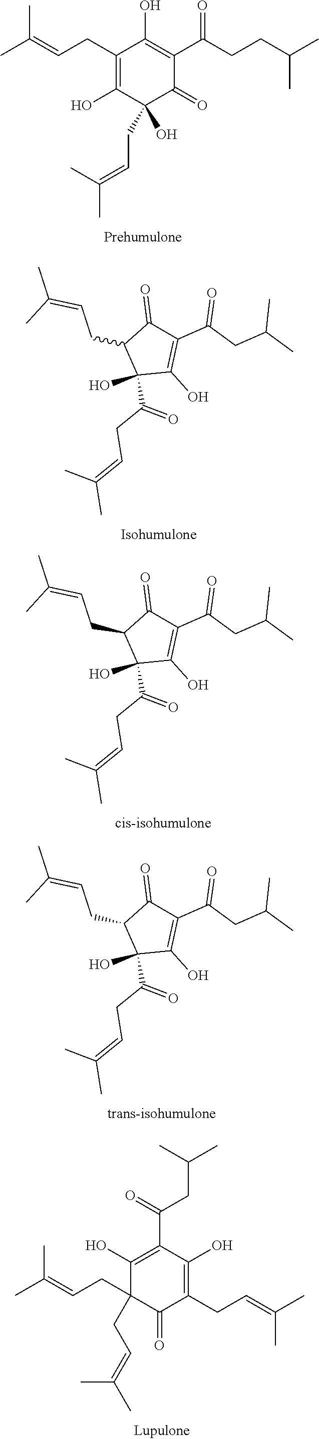

α-acid and/or β-acid as used in the present invention may be a commercially available pure form of the molecule, or alternatively be an (enriched) extract or a matured hop extract obtained from a suitable source such as Humulus.

Furthermore, it is noted that in this application Humulus may also be referred to as hop.

It is also noted that wt. % mentioned in this application refer to weight percentage of the dry weight of the composition according to the invention.

A particular advantage of the composition according to the invention is that it has a high bioavailability and hence increased efficacy.

A further advantage of the composition according to the invention is that the solubility of the composition is up to 100 g L-1 at 19° C. and a pH of 7.

A further advantage of the composition and process according to the invention is that the acids are maintained in their acidic state, thereby maintaining the original characteristics of the acids.

A further advantage is that an efficient and effective composition in the treatment and/or cure and/or prevention of suppression of inflammatory responses and attenuate neural hyperactivation in the hippocampus, immunomodulatory activity, and metabolic syndromes, against obesity and fatty liver disease, irritable bowl syndrome, improved sleep induction, reduction of anxiety levels, reduction of stress levels, and reduction of depression symptoms is achieved.

Furthermore, it was found that the composition according to the invention decreases methane production in rumen, provides positive effects on body weight gain and intestinal tract in animals (eg. cattle pigs, poultry), and reduces biofilm formation. It was also found that the composition according to the invention comprising (semi-synthetic) α-acids and/or a β-acids enables efficient and effective prevention of coccidiosis. Therefore, said composition may be included in a feed supplement.

The compositions of the present invention are made using an extrusion process, which in general comprises at least two steps including dry mixing and extrusion. First, the materials are dry mixed to achieve a homogeneous powder. The blend is hot-melt extruded and the extrudate may subsequently be milled or reduced in size by any other means. Where applicable, the extrudates or size-reduced particles obtained therefrom are used in the formulations of the present invention. The resulting end products of such an extrusion process (prior to size-reduction) according to the present invention, are termed ‘extrudates’. Hence, the compositions of the present invention are characterized in being in the form of an extrudate, i.e. by being produced using the described extrusion process.

It was surprisingly found that a thermoplastic polymer could significantly improve the solubility of the α-acid and/or the β-acid in a hot-melt extrusion process (e.g. improves the solubility of the α-acid and/or the β-acid in cellulose and/or copolymer). Furthermore, the extrudate may be soluble in water, including the α-acid and/or the β-acid. Such thermoplastic polymer is preferably nutritionally and/or cosmetically acceptable and/or therapeutically acceptable and/or pharmaceutically acceptable, depending on the type of formulation to be used in, e.g. nutritionally acceptable for oral formulations, cosmetically and/or therapeutically and/or nutraceutical and/or pharmaceutically acceptable for topical formulations. More specifically, such thermoplastic polymers are preferably “GRAS” (Generally Recognized/Regarded As Safe) components. Generally recognized as safe (GRAS) is an American Food and Drug Administration (FDA) designation that a chemical or substance added to food is considered safe by experts, and so is exempted from the usual Federal Food, Drug, and Cosmetic Act (FFDCA) food additive tolerance requirements.

In the context of the present invention, the term “thermoplastic polymer” is meant to be a polymer/material which is pliable or mouldable above a specific temperature and which becomes solid again upon cooling. Such thermoplastic polymers are in particular highly suitable for use in a hot-melt extrusion process as used in the current invention.

Suitable thermoplastic polymers within the context of the invention may for example be selected from the list comprising homopolymers and copolymers of N-vinyl lactams, cellulose derivatives, high molecular polyalkylene oxides, polyvinyl alcohol-polyethylene glycol-graft copolymers, graft copolymers comprising a poly(alkylene glycol) backbone and a vinyl acetate/N-vinylcaprolactam copolymer grafted onto the backbone, polyacrylates and polymethacrylates, polyacrylamides, vinyl acetate polymers, polyvinyl alcohol, oligo- and polysaccharides, polyhydroxyalkanoates, polyamino acids, proteins and polypeptides and mixtures thereof. In particular, the thermoplastic polymers may be selected from the list comprising hydroxypropylmethylcellulose (HPMC), a vinylpyrrolidone-vinyl acetate copolymer, Eudragit E and mixtures thereof. More in particular, the thermoplastic polymer of the present invention may be hydroxypropylmethylcellulose (HPMC), a vinylpyrrolidone-vinyl acetate copolymer, or a combination thereof.

HPMC as used in the current invention is meant to be hydroxypropylmethylcellulose or hypromellose and is represented by the following general formula:

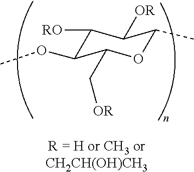

Vinylpyrrolidone-vinyl acetate copolymer as used herein is meant to be a copolymer of (poly) vinylpyrrolidone (PVP) and vinyl acetate (VA), each represented by the appropriate general formula:

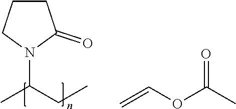

### PVP Vinyl Acetate

Both components may be combined to a co-polymer using any suitable ratio. In that respect several different combinations are marketed for example under the tradename Kollidon®. A particularly suitable combination within the context of the present invention is Kollidon® VA64, which is a copovidone having the CAS registration N° 25086-89-9. It is defined as being a vinylpyrrolidone-vinyl acetate copolymer in a ratio of 6:4 by mass and represented by the following general formula:

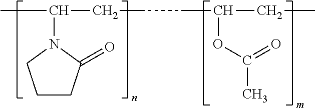

In a certain embodiment, the composition according to the invention comprises the thermoplastic polymer in the range of 50 wt. % and 90 wt. %, preferably in the range of 60 wt. % and 90 wt. %, more preferably comprises the thermoplastic polymer in an amount of about 90 wt. %, at least 85 wt. %, at least 80 wt. %, at least 75 wt. %, at least 70 wt. %, at least 65 wt. %, or at least 60 wt. %.

In a preferred embodiment according to the invention, the composition of the present invention comprises a plasticizer. Preferably, wherein the plasticizer is polyethylene glycol and/or polyglyceryl-3 polyricinoleate.

It was found that the composition comprising a plasticizer improved the ability to perform an hot-melt extrusion. In addition, it was found that the composition comprising a plasticizer enabled to improve the solubility of the α-acid and/or the β-acid in the cellulose and/or copolymer. As a result, an efficient and effective hot-melt extrudate has been achieved.

Furthermore, it is noted that the composition without a plasticizer must be extruded at higher temperatures, in order to achieve the desired composition. Therefore, degradation of the α-acid and/or the β-acid may occur and a higher energy conception is required.

It was found that when the plasticizer is polyethylene glycol and/or polyglyceryl-3 polyricinoleate, an efficient and effective hot-melt extrudate has been achieved and the solubility of the α-acid and/or the β-acid has been improved as the extrusion may be performed at a temperature in the range of 50° C. to 200° C., preferably in the range of 80° C. to 170° C., more preferably in the range of 80° C. to 150° C.

In a preferred embodiment, the plasticizer is polyethylene glycol, wherein the polyethylene glycol has a molecular weight in the range of 400 g mol-1 to 8000 g mol-1.

In a further preferred embodiment, the plasticizer is polyglyceryl-3 polyricinoleate. Furthermore, it is noted that the polyglyceryl-3 polyricinoleate may be used in combination with the polyethylene glycol.

In addition, the plasticizer and the thermoplastic polymer are preferably present in a ratio between 1:10 to 1:25, more preferably in a ratio between 1:15 to 1:20. Furthermore, the ratio between plasticizer and α-acid and/or β-acid is in the range of 1:1 to 1:6, preferably in the range of 1:1 to 1:4, more preferably in the range of 1:1 to 1:2.

In a further preferred embodiment, the plasticizer may be present at a concentration in the range of 0.5 wt % and 30 wt % with respect to the polymer, more preferably in the range of 10 wt. % and 30 wt. %, such as at least 10 wt. %, at least 15 wt. %, at least 20 wt. %, at least 25 wt. % or about 30 wt. %.

In a further preferred embodiment according to the invention, the α-acid is present in the range of 0.5 wt. % to 50 wt. % of the dry weight of the composition, preferably in the range of 0.5 wt. % to 30 wt. % of the dry weight of the composition.

In particular, the α-acid is present at a concentration of at least 10 wt. % of the dry weight of the composition, in particular in the range of 10 wt. % to 50 wt. % of the dry weight of the composition, such as in the range of 10 wt. % to 30 wt. % of the dry weight of the composition.

Preferably, the composition according to the invention comprises at least 0.5 wt. %, at least 1 wt. %, at least 2 wt. %, at least 3 wt. %, at least 4 wt. %, at least 5 wt. %, at least 6 wt. %, at least 7 wt. %, at least 8 wt. %, at least 9 wt. %, at least 10 wt. %, at least 15 wt. %, at least 20 wt. %, at least at least 25 wt. %, or at least 30 wt. % of the α-acid.

In yet a further preferred embodiment according to the invention, the β-acid is present in the range of 0.5 wt. % to 50 wt. % of the dry weight of the composition, preferably in the range of 0.5 wt. % to 30 wt. % of the dry weight of the composition.

In particular, the β-acid is present at a concentration of at least 10 wt. % of the dry weight of the composition, in particular in the range of 10 wt. % to 50 wt. % of the dry weight of the composition, such as in the range of 10 wt. % to 30 wt. % of the dry weight of the composition.

Preferably, the composition according to the invention comprises at least 0.5 wt. %, at least 1 wt. %, at least 2 wt. %, at least 3 wt. %, at least 4 wt. %, at least 5 wt. %, at least 6 wt. %, at least 7 wt. %, at least 8 wt. %, at least 9 wt. %, at least 10 wt. %, at least 15 wt. %, at least 20 wt. %, at least 25 wt. %, or at least 30 wt. % of the β-acid.

It was found that the α-acid and/or the β-acid present in the aforementioned amount, provides an efficient and effective composition.

In yet a further preferred embodiment according to the invention, the α-acid is an iso-α-acid. Preferably, the iso-α-acid is a tetra-hydro-iso-α-acid and/or a hexa-hydro-iso-α-acid.

It was found that, in particular, an iso-α-acid, preferably a tetra-hydro-iso-α-acid and/or a hexa-hydro-iso-α-acid, provides a composition which is stable and suitable for use in an aqueous environment.

In yet a further preferred embodiment according to the invention, the α-acid is one or more selected from the group of humulone, adhumulone, cohumulone, posthumulone, and prehumulone, preferably the α-acid is cis-iso-humulone and/or trans-iso-humulone and/or the β-acid is lupulone.

It was found that the α-acid and/or the β-acid being one or more selected from the aforementioned groups provides efficient and effective suppression of inflammatory responses and attenuate neural hyperactivation in the hippocampus, immunomodulatory activity, and metabolic syndromes against obesity and fatty liver disease, irritable bowl syndrome, improved sleep induction, reduction of anxiety levels, reduction of stress levels, and reduction of depression symptoms is achieved.

Furthermore, it was found that the composition according to the invention decreases methane production in rumen, provides positive effects on body weight gain and intestinal tract in animals (eg. cattle, pigs, poultry), and reduces biofilm formation. In addition, it was also found that the composition according to the invention comprising (semi-synthetic) α-acids and/or a β-acids enables efficient and effective prevention of coccidiosis.

Accordingly, in a specific embodiment, the present invention provides the compositions as defined herein for use in the prevention and/or treatment of coccidiosis in a subject in need thereof.

In yet a further preferred embodiment according to the invention, the composition further comprises xanthohumol, preferably xanthohumol extrudate.

In the context of the present invention the term “xanthohumol” is to be understood as meaning a prenylated chalconoid obtainable from hop and beer, and is represented by the following formula:

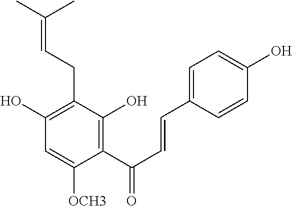 [SMILES: COc1cc(O)c(CC=C(C)C)c(O)c1C(=O)/C=C/c1ccc(O)cc1]

Xanthohumol as used in the present invention may be a commercially available pure form of the molecule, or alternatively be an (enriched) extract obtained from a suitable source such as hop.

In yet a further preferred embodiment according to the invention, the composition further comprises one or more additive selected from the group of microcrystalline cellulose, lactose, dextrose, sucrose, sorbitol, mannitol, maltodextrin, di basic calcium phosphate, cellulose, pregelatinized starch, croscarmellose sodium, crospovidone, sodium starch glycolate, calcium carbonate, magnesium carbonate, alginic acid, hydroxypropylmethylcellulose, hydroxypropylcellulose, soy polysaccharide, xylan, xanthan gum, aluminium silicate, magnesium silicate, colloidal silica, aluminium stearate, calcium stearate, magnesium stearate, zinc stearate, magnesium lauryl sulphate, sodium lauryl sulphate, sodium stearyl fumarate, glyceryl tristearate, glyceryl monostearate, glyceryl behenate, palmitic acid, stearic acid, palmitoyl alcohol, stearyl alcohol, hydrogenated vegetable oil, hydrogenated castor oil, mineral oil, fumaric acid, starch, talc, sodium benzoate, calcium silicate, magnesium silicate, magnesium oxide, colloidal silicon dioxide, cellulose, starch, talc.

It was found that one or more selected from the group of microcrystalline cellulose, lactose, dextrose, sucrose, sorbitol, mannitol, maltodextrin, di basic calcium phosphate, cellulose, pregelatinized starch may be used as binder and/or filler of the composition according to the invention.

Furthermore, it was found that one or more selected from the group of croscarmellose sodium, crospovidone, sodium starch glycolate, calcium carbonate, magnesium carbonate, alginic acid, hydroxypropylmethylcellulose, hydroxypropylcellulose, (soy) polysaccharide, xylan, xanthan gum, magnesium aluminium silicate may be used as disintegrant in the composition according to the invention.

In addition, it was found that one or more selected from the group of aluminium stearate, calcium stearate, magnesium stearate, zinc stearate, magnesium lauryl sulphate, sodium lauryl sulphate, sodium stearyl fumarate, glyceryl tristearate, glyceryl monostearate, glyceryl behenate, palmitic acid, stearic acid, palmitoyl alcohol, stearyl alcohol, hydrogenated vegetable oil, hydrogenated castor oil, mineral oil, fumaric acid, starch, talc, sodium benzoate, may be used as lubricant in the composition according to the invention.

Furthermore, it was found that one or more selected from the group of calcium silicate, magnesium silicate, magnesium oxide, colloidal silicon dioxide, cellulose, starch, talc, may be used as glidant in the composition according to the invention.

In yet a further preferred embodiment according to the invention, the α-acid and/or the β-acid originate from Humulus lupulus and/or are semi-synthetic α-acid and/or semi-synthetic β-acid.

It is noted that Humulus lupulus may also be referred to as ‘common’ hops.

An advantage of the α-acid and/or the β-acid originating from Humulus lupulus is that said α-acid and/or said β-acid are formed in an environmental friendly manner.

In yet a further preferred embodiment according to the invention, the extrudate is in the form of an extrudate as such, a powder, a nanoparticulate form or a microparticulate form. Such forms of the composition may be obtained for example by milling the extrudates to obtain the desired particle size. However, any other method suitable for obtaining such forms from the extrudates may be used.

As used herein, the term “powder” is meant to be a solid substance which is reduced to a state of fine, loose particles (i.e. multiparticulate) such as for example by crushing, grinding or milling. As used herein, “multiparticulate” means a plurality of discrete, or aggregated, particles, extrudates, pellets, beads, granules or mixture thereof irrespective of their size, shape or morphology. By way of example each particle may have a diameter of from about 0.1 mm to about 5.0 mm, e.g. from about 0.5 mm to about 5.0 mm, in particular from about 0.5 mm to about 2.5 mm, more particularly from about 0.5 mm to about 1.0 mm, more in particular about 0.75 mm. The average size of the individual powder particles (i.e. multiparticulate) determines its eventual nomenclature. For example, “nanoparticulate” refers to a multiparticulate in which the effective average particle size of the particles therein is less than 1 μm in diameter. On the other hand, “microparticulate” refers to a multiparticulate in which the effective average particle size of the particles therein is between 1 nm and 1 mm, preferably between 1 μm and 1 mm in diameter.

In a further preferred embodiment, the composition further comprises a tensio-active agent. Whenever a tensio-active agent is present, it is usually present at a maximum concentration of 20 wt. % of the total composition, such as at most 15 wt. %, at most 10 wt. %, at most 5 wt. %, at most 4 wt. %, at most 3 wt. %, at most 2 wt. %, at most 1 wt. %.

The invention also relates to a topical formulation comprising the composition according to the invention. Preferably, the topical formulation is a topical cosmetic formulation and/or a topical pharmaceutical formulation and/or a topical therapeutic formulation and/or topical nutraceutical formulation.

The topical formulation provides the effects and advantage as the composition according to the invention.

To provide the topical formulation, the extrudates are further processed to a format suitable for incorporation in a topical formulation. For example, the extrudates may be milled to a powder, which can then be included in a standard or custom-made topical formulation, for example a custom-made topical cosmetic formulation and/or a custom-made topical pharmaceutical formulation and/or a custom-made topical therapeutical formulation and/or custom-made topical nutraceutical formulation, such as for example in a gel, cream, foam, paste, lotion, milk, emulsion, solution, suspension, ointment, lipstick, shower gel, bath gel, shampoo, sunscreen, after sun preparation, spray, moisturizer, anti-dandruff formulation, antiperspirant or deodorant composition. Evidently, any other form of topical formulation and/or oral formulation which allows the inclusion of the extrudates or further processed forms derived thereof, are also envisaged within the context of the current invention.

In a further preferred embodiment according to the invention, the topical formulation may further comprise one or more additional components such as selected from the list comprising: hydration agents, vitamins, antioxidants, peptides, plant extracts, anti-slimming components, anti-ageing components, anti-acne components, anti-inflammatory components, sleep inducing agents, metabolic agents and cognitive improving agents.

The invention also relates to an oral formulation comprising the composition according to the invention.

It is noted that the oral formulation may refer to therapeutical formulations and/or pharmaceutical formulations and/or nutraceutical formulations.

The oral formulation provides the same effects and advantages as the composition according to the invention and the topical formulation according to the invention.

While the invention is in particular suitable for formulating an α-acid and/or a β-acid in aqueous formulations, it may also be suitably used in the formulation of an α-acid and/or a β-acid in formulations for oral use. Thereto, the present invention also provides an oral formulation comprises the compositions as described herein. Such oral formulation may for example be in the form of a pharmaceutical formulation, a therapeutical formulation, a nutraceutical formulation, a food supplement, or a feed supplement. For example, the feed supplement according to the invention may be used in the prevention of coccidiosis. In addition, the oral formulation may be provided as (grinded) extrudate to (animal) feed.

Pharmaceutical formulations as used herein are formulations including the compositions of the invention in combination with other pharmaceutically acceptable compounds such as excipients, carriers, and the like.

Nutraceutical formulations as used herein are formulations that include the compositions of the invention in combination with natural ingredients and/or supplements that promote good health.

Food supplement as used herein contain the compositions of the invention in an edible carrier (food product). Examples of an edible carrier include starch, cyclodextrin, maltodextrin, methylcellulose, carboxymethylcellulose, xanthan gum, and aqueous solutions thereof. Such food products can be prepared by methods well known in the food industry. As used herein, the term “food” broadly refers to any kinds of liquid and solid/semi-solid materials that are used for nourishing humans and animals.

In a preferred embodiment according to the invention, this oral formulation is in the form of an extrudate, a powder, uncoated or coated capsule, uncoated or coated tablet, pill, lozenge, sachet, cachet, elixir, suspension, emulsion, solution, syrup, uncoated or coated soft and hard gelatin capsules, granule and uncoated or coated pellet.

In a further particular embodiment, the present invention provides an oral formulation as defined herein, wherein said formulation further comprises one or more components selected from the list comprising: hydration agents, vitamins, antioxidants, peptides, plant extracts, anti-slimming components, anti-ageing components, anti-acne components, anti-inflammatory components.

The invention also relates to the use of the composition according to the invention and/or the oral formulation according to the invention in human and/or veterinary application; or alternatively in topical formulations; or alternatively in cosmetics.

The use of the composition according to the invention and/or the oral formulation according to the invention in human and/or veterinary application; or alternatively in topical formulations; or alternatively in cosmetics provides the same effects and advantages as the composition according to the invention, the topical formulation according to the invention, and the oral formulation according to the invention.

The invention also relates to a method for the preparation of a composition according to the invention, comprising the steps of:
mixing an α-acid and/or a β-acid from hop and/or a semi-synthetic α-acid and/or a semi-synthetic β-acid with hydroxypropylmethylcellulose and/or vinylpyrrolidone-vinyl acetate copolymer;
hot-melt extruding said mixture; and
optionally milling said hot-melt extruded mixture.

The method for the preparation of a composition according to the invention provides the same effects and advantages as the composition according to the invention, the topical formulation according to the invention, the oral formulation according to the invention, and use of the composition according to the invention and/or the oral formulation according to the invention.

In a preferred embodiment according to the invention, the α-acid is an iso-α-acid. Preferably, the iso-α-acid is a tetra-hydro-iso-α-acid and/or a hexa-hydro-iso-α-acid.

In a further preferred embodiment according to the invention, the method further comprises the step of mixing further comprises mixing a plasticizer and/or xanthohumol with the α-acid and/or the β-acid and/or the semi-synthetic α-acid and/or the semi-synthetic β-acid and hydroxypropylmethylcellulose and/or vinylpyrrolidone-vinyl acetate copolymer.

The invention also relates to the use of hydroxypropylmethylcellulose and/or vinylpyrrolidone-vinyl acetate copolymer in a hot-melt extrusion process of an α-acid and/or a β-acid from Humulus and/or an α-acid and/or a β-acid as a semi-synthetic form, preferably in a hot-melt extrusion process of an iso-α-acid from Humulus and/or an iso-α-acid as a semi-synthetic form.

The use of hydroxypropylmethylcellulose and/or vinylpyrrolidone-vinyl acetate copolymer in a hot-melt extrusion process of an α-acid and/or a β-acid from Humulus and/or an α-acid and/or a β-acid as a semi-synthetic form provides the same effects and advantages as the composition according to the invention, the topical formulation according to the invention, the oral formulation according to the invention, use of the composition according to the invention and/or the oral formulation according to the invention, and the method for the preparation of a composition according to the invention.

### EXAMPLES

The tetra-hydro-iso-α-acids extrudate was prepared using the following general process: a mixture of Kollidon VA64, PEG 1500 and tetra-hydro-iso-α-acids (85:5:10) was hot-melt extruded using a corotating twin extruder at a screw speed of 200 rpm at 130° C. The thermoplastic extrudate was collected, cooled to room temperature and milled to obtain a powder, which was then used as such in the below mentioned examples

### Tablet Formulation

| Composition | Amount g |
|---|---|
| tetra-hydro-iso-α-acids milled extrudate (10%) | 250.0 |
| Microcrystalline cellulose | 469.0 |
| Colloidal silicium dioxide | 1.0 |
| Sodium stearyl fumarate | 5.0 |
| Cross-linked polyvinylpyrrolidone | 25.0 |

### Soluble Powder for Drinking Water Medication or Powder as Premix for Feed Medication

| Composition | Amount (g) |
|---|---|
| tetra-hydro-iso-α-acids milled extrudate (10%) | 15.0 |
| Xanthohumol milled extrudate | 13.3 |
| Colloidal silicium dioxide | 0.5 |
| Mannitol or maltodextrine | 26.2 |

### Cosmetic Cream Formulation

| Composition | Amount (g) | |
|---|---|---|
| Cetostearylalcohol | 4 | |
| Ceteareth 20 | 1.5 | |
| Isopropylpalmitaat | 3 | |
| Lipocire A | 1 | |
| Carbopol 980 | 0.15 | |
| Tocopherolacetaat | 1 | |
| Potassium sorbate | 0.27 | |
| tetra-hydro-iso-α-acids milled extrudate (10%) | 0.05 | |
| Sodium hydroxide solution 1M | 0.5 | g |
| Water | 88.53 | g |

### Example 1: Effect on the Growth and Viability of in In Vitro Cultivated Histomonas meleagridis

The effect of a tetra-hydro-iso-α-acids milled extrudate on the growth of Histomonas meleagridis in vitro was investigated.

Therefore, a milled extrudate of tetra-hydro-iso-α-acids (corresponding to an amount of 200 μg tetra-hydro-iso-α-acid/ml), a positive control (dimetridazole, concentration of 400 μg/ml) and a negative control (distilled water without addition of any substance) were used.

Histomonas meleagridis cells were counted before and after 24, 48 and 72 hours of incubation.

| | Time of incubation | | | |
|---|---|---|---|---|
| Substance | 0 h | 24 h | 48 h | 72 h |
| Negative control group | 1 × 10⁵ | 8.30 × 10⁴ | 1.48 × 10⁵ | 1.93 × 10⁵ |
| No substance | | | | |
| Positive control group | 1 × 10⁵ | 0 | 0 | 0 |
| Dimetridazole (400 μg/ml) | | | | |
| tetra-hydro-iso-α-acids | 1 × 10⁵ | 0 | 0 | 0 |
| milled extrudate | | | | |
| (200 μg/ml) | | | | |

A total reduction of Histomonas meleagridis cells was obtained after addition of dimetridazole and iso-α-acids, preferably tetra-hydro-iso-α-acids, administered as milled extrudate, while no effect was observed in the negative control group.

### Example 2: Efficacy Against Histomonas meleagridis

To investigate the efficacy of a tetra-hydro-iso-α-acids milled extrudate on Histomonas meleagridis, 14-days old turkeys were infected with 1×10⁵ Histomonas meleagridis cells.

From day 0 until the end of the experiment, the tetra-hydro-iso-α-acids milled extrudate (800 mg/kg feed) was administered as such and in combination with a xanthohumol (XA) milled extrudate (100 mg/kg feed). Besides, a mixture of tetra-hydro-iso-α-acids as such (no extrusion) and xanthohumol as such (no extrusion) was administered from day 0 until the end of the experiment.

A negative control group (no infection, no treatment) and an infection control group (infection, no treatment) were also enclosed.

The percentage of infected turkeys was determined and 10 days after infection, the presence of disease signs in liver and caeca was evaluated on a scale from 0 (no lesions) to 4 (severe lesions).

| | Infected | Liver | Caeca |
|---|---|---|---|
| Treatment | turkeys | scores | scores |
| Negative control group | 0% | 0 | 0 |
| (no treatment, no infection) | | | |
| Infection control group | 100% | 3.6 | 3.2 |
| (no treatment, infection) | | | |
| tetra-hydro-iso-α-acids milled extrudate | 56% | 2.0 | 0.9 |
| (800 mg/kg feed) | | | |
| tetra-hydro-iso-α-acids milled extrudate | 20% | 0.7 | 0.4 |
| (800 mg/kg feed) + | | | |
| XA milled extrudate (100 mg/kg feed) | | | |
| tetra-hydro-iso-α-acids (4800 mg/kg | 100% | 3.5 | 3.2 |
| feed) + XA (100 mg/kg feed) | | | |

The use of a tetra-hydro-iso-α-acids milled extrudate results in a decreased degree of infection and lower liver and caeca scores. The effect is even more pronounced if the tetra-hydro-iso-α-acids extrudate was used in combination with a xanthohumol milled extrudate. No differences compared to the infection control group were observed if tetra-hydro-iso-α-acids and xanthohumol were administered without extrusion. Accordingly, these data evidence the fact that the extruded compositions of the present invention have a higher bioavailability compared to non-extruded compositions.

### Example 3: Efficacy Against Eimeria Species in Broiler Chickens

To investigate the efficacy of a tetra-hydro-iso-α-acids milled extrudate on different Eimeria species in broiler chickens, 14-days old broilers were infected with a mixed suspension of 2.5×10⁴ sporulated E. acervulina and E. tenella oocysts.

From day 0 until the end of the experiment, the tetra-hydro-iso-α-acids milled extrudate (400 or 800 mg/kg feed, respectively) was administered in combination with a xanthohumol (XA) milled extrudate (50 and 100 mg/kg feed, respectively).

A negative control group (no infection, no treatment), a positive control group (infection, no treatment) and a group treated with a reference product (infection-treatment with Sacox 120®-Salinomycin 70 ppm from day 0 until the end of the experiment) were also enclosed.

The lesions in duodenum and caeca, the percentage of the reduction of the oocysts per gram faeces (OPG) and the anticoccidial index (ACI) (parameter of effectiveness to prevent coccidiosis-based on OPG, lesions and body weight gain) were determined.

| | | | Evolution | |
|---|---|---|---|---|
| | Lesions | Lesions | OPG | |
| Treatment | duodenum | caeca | reduction | ACI |
| Negative control group | 0 | 0 | n.d. | 0 |
| (no treatment, no infection) | | | | |
| Positive control group | 2.0 | 2.5 | n.d. | 60 |
| (no treatment, infection) | (1.0-2.0) | (2.0-3.0) | | |
| Reference product | 1.0 | 2.0 | −37% | 122 |
| Sacox 120 ® | (1.0-1.5) | (2.0-3.0) | | |
| (Salinomycin 70 ppm) | | | | |
| tetra-hydro-iso-α-acids milled | 1.0 | 1.0 | −24% | 134 |
| extrudate (400 mg/kg feed) + | (1.0-1.5) | (0.5-2.0) | | |
| XA milled extrudate | | | | |
| (50 mg/kg feed) | | | | |
| tetra-hydro-iso-α-acids milled | 1.5 | 2.0 | +19% | 27 |
| extrudate (800 mg/kg feed) + | (0.5-2.0) | (2.0-3.0) | | |
| XA milled extrudate | | | | |
| (100 mg/kg feed) | | | | |

The use of an tetra-hydro-iso-α-acids milled extrudate at a dose of 400 mg/kg feed in combination with a xanthohumol milled extrudate at a dose of 50 mg/kg feed results in a lower lesion scores in duodenum and caeca and a better anticoccidial index compared to a widely used reference product (Sacox 120®, Salinomycin 70 ppm).

The present invention is by no means limited to the above described preferred embodiments and/or experiments thereof. The rights sought are defined by the following claims within the scope of which many modifications can be envisaged.

### Example 4: Evaluation of the Solubility of the Compositions of the Invention

In this example, we compared the solubility of a composition as prepared according to the process of the present invention, with that of a composition as prepared according to a prior art process as disclosed in WO2009/142736. For comparative reasons, in both processes the same extract was used.

### Materials and Methods

Preparing Lupuhop® spray-dried powder according to WO 2009/142736 (herein '736) 4 g of Lupuhop® (BarthHaas, Germany), a modified hop extract containing 58% iso-alpha-acids was dispersed in 50 ml water. The pH of this dispersion was measured and a value of 3.53 was registered. Then, pH was adjusted to 8.94 by using sodium hydroxide 1M. The solution was filtered and after addition of 4 g maltodextrin (C*PharmDry 01983), the solution was spray dried to obtain a powder.

### Preparing Lupuhop® Extrudate According to the Present Invention

The extrudate was prepared using the following process: a mixture of Lupuhop® (BarthHaas, Germany), PEG 1500 and Kollidon® VA64 (BASF, Germany) was hot-melt extruded using a corotating twin screw extruder at a screw speed of 200 rpm at 130° C. The extrudate was collected, cooled and milled to obtain a powder.

### Results

### Lupuhop® Spray-Dried Powder According '736

100 mg of the spray-dried powder as obtained according to the process of D2 was dispersed in 30 ml of demineralized water. This amount of spray-dried powder contains 50 mg Lupuhop® (the spray-dried powder contains 50% Lupuhop® and 50% maltodextrins).

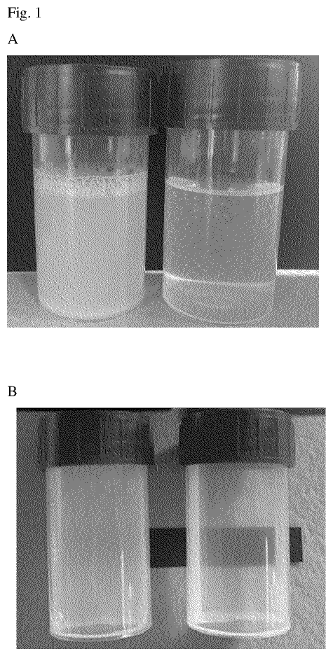

A turbid dispersion of the product was obtained indicating that the product is not dissolved but finely dispersed in the water (see FIGS. 1A and B—left panels).

### Lupuhop® Extrudate According to the Present Invention

500 mg of the milled (powdered) extrudate was dispersed in 30 ml of demineralized water. This amount of milled extrudate contains 50 mg Lupuhop® (the extrudate contains 10% Lupuhop®, 5% PEG 1500 and 85% Kollidon® VA64).

A clear solution of the product was obtained indicating that the product is dissolved and not dispersed in the water (see FIGS. 1A and B, right panels).

As evident from FIGS. 1A and 1B, the formulation containing the milled extrudate (right) is clear whereas, the formulation with the spray-dried (SD) powder (left) is dispersed and not dissolved. This is very apparent from FIG. 1B, wherein the black strip behind the flasks is clearly visible only in the formulation prepared according to the invention.

### CONCLUSIONS

The Lupuhop® formulation obtained after milling the extrudate using the process of the invention, results in a clear solution in water, despite Lupuhop® itself is not soluble in water. Furthermore, our process does not require pH adjustment for improving solubility. In contrast without any pH adjustment the process of the invention obtains a highly soluble product, whereas in '736 even after pH adjustment for improving solubility, still a product which only disperses in water is obtained. Therefore, our process provides a solution to the problem of insolubility of hop acids in water, whereas '736 fails in doing so. Since pH adjustment also affects the acid form of hop acids, we even provide a product in which the original characteristics of the hop acids are maintained in contrast to '736 in which sodium salts of such acids are made.

Therefore, the extrudate process of the present invention results in products which are clearly distinguishable from the products obtained by the prior art process of '736. Moreover, these products have significant benefits in terms of solubility and maintenance of acid characteristics over the products of the prior art.

1. Composition comprising an α-acid and/or a β-acid from Humulus and a hydroxypropylmethylcellulose and/or a vinylpyrrolidone-vinyl acetate copolymer, wherein said composition is in the form of an hot-melt extrudate; wherein the α-acid is present at a concentration of at least 10 wt. % of the dry weight of the composition, and/or wherein the β-acid is present at a concentration of at least 10 wt. % of the dry weight of the composition of the dry weight of the composition.

2. Composition according to claim 1, further comprising a plasticizer, preferably wherein the plasticizer is selected from the list comprising polyethylene glycol (PEG), polyglyceryl-3 polyricinoleate and combinations thereof.

3. Composition according to any one of the preceding claims, wherein the α-acid is present in the range of 10 wt. % to 50 wt. % of the dry weight of the composition, preferably in the range of 10 wt. % to 30 wt. % of the dry weight of the composition, and/or wherein the β-acid is present in the range of 10 wt. % to 50 wt. % of the dry weight of the composition, preferably in the range of 10 wt. % to 30 wt. % of the dry weight of the composition.

4. Composition according to any one of the preceding claims, wherein the α-acid is an iso-α-acid, preferably wherein the iso-α-acid is a tetra-hydro-iso-α-acid and/or a hexa-hydro-iso-α-acid.

5. Composition according to any one of the preceding claims, wherein the α-acid is one or more selected from the group of humulone, adhumulone, cohumulone, posthumulone, and prehumulone, preferably the α-acid is cis-iso-humulone and/or trans-iso-humulone, and/or wherein the β-acid is lupulone.

6. Composition according to any one of the preceding claims, further comprising xanthohumol, preferably xanthohumol extrudate, and/or further comprising one or more additive selected from the group of microcrystalline cellulose, lactose, dextrose, sucrose, sorbitol, mannitol, maltodextrin, di basic calcium phosphate, cellulose, pregelatinized starch, croscarmellose sodium, crospovidion, sodium starchglycolate, calciumcarbonate, magnesiumcarbonate, alginic acid, hydroxypropylcellulose, hydroxypropylcellulose, soy polysaccharide, xylan, xanthan gum, aluminium silicate, magnesium silicate, colloidal silica, aluminium stearate, calcium stearate, magnesium stearate, zinc stearate, magnesium lauryl sulphate, sodium lauryl sulphate, sodium stearyl fumarate, glyceryl tristearate, glyceryl monostearate, glyceryl behenate, palmitic acid, stearic acid, palmitoyl alcohol, stearyl alcohol, hydrogenated vegetable oil, hydrogenated castor oil, mineral oil, fumaric acid, starch, talc, sodium benzoate, calcium silicate, magnesium silicate, magnesium oxide, colloidal silicon dioxide, cellulose, starch, talc.

7. Composition according to any one of the preceding claims, wherein the α-acid and/or the β-acid originate from Humulus lupulus and/or are in the form of a Humulus extract, in semi-synthetic form or in a salt form thereof, either or not fixed on a carrier, and/or wherein the extrudate is in the form of an extrudate as such, a powder, a nanoparticulate form or a microparticulate form.

8. A topical formulation comprising the composition according to any one of the preceding claims.

9. The topical formulation according to claim 8, wherein said formulation is in the form of a gel, cream, foam, paste, lotion, milk, emulsion, solution, suspension, ointment, lipstick, shower gel, bath gel, shampoo, sunscreen, after sun preparation, spray, moisturizer, anti-dandruff formulation, antiperspirant or deodorant composition, and/or wherein said formulation further comprises one or more components selected from the list comprising: hydration agents, vitamins, antioxidants, peptides, plant extracts, anti-slimming components, anti-ageing components, anti-acne components, anti-inflammatory components, stimulants, sleep inducing agents, metabolic agents and cognitive improving agents.

10. An oral formulation comprising the composition according to any one of the claims 1 to 7.

11. The oral formulation according to claim 10, which is in the form of a pharmaceutical formulation, a therapeutical formulation, a nutraceutical formulation, a food supplement, a feed supplement, and/or wherein said formulation is in the form of an extrudate, a powder, uncoated or coated capsule, uncoated or coated tablet, pill, lozenge, sachet, cachet, elixir, suspension, emulsion, solution, syrup, uncoated or coated soft and hard gelatin capsules, granule or uncoated or coated pellet; and/or wherein said formulation further comprises one or more components selected from the list comprising: hydration agents, vitamins, antioxidants, peptides, plant extracts, anti-slimming components, anti-ageing components, anti-acne components, anti-inflammatory components, stimulants, sleep inducing agents, metabolic agents and cognitive improving agents.

12. The composition according to any one of the claims 1 to 7; and/or the topical formulation according to any one of claim 8 or 9; and/or the oral formulation according to any one of the claim 10 or 11 for use in human and/or veterinary application.

13. A method for the preparation of a composition according to any one of the claims 1 to 7, comprising the steps of:
mixing an α-acid and/or a β-acid as defined in any of the previous claims with hydroxypropylmethylcellulose and/or vinylpyrrolidone-vinyl acetate copolymer; wherein the α-acid is used at a concentration of at least 10 wt. % of the dry weight of the composition, and/or wherein the β-acid is used at a concentration of at least 10 wt. % of the dry weight of the composition of the dry weight of the composition
hot-melt extruding said mixture; and
optionally milling said hot-melt extruded mixture.

14. Method according to claim 13, wherein the α-acid is an iso-α-acid, preferably wherein the iso-α-acid is a tetra-hydro-iso-α-acid and/or a hexa-hydro-iso-α-acid, and/or wherein the step of mixing further comprises mixing a plasticizer and/or xanthohumol with said α-acid and/or the β-acid and hydroxypropylmethylcellulose and/or vinylpyrrolidone-vinyl acetate copolymer.

15. Use of hydroxypropylmethylcellulose and/or vinylpyrrolidone-vinyl acetate copolymer in a hot-melt extrusion process of an α-acid and/or a β-acid as defined in any of the previous claims, preferably in a hot-melt extrusion process of an iso-α-acid from Humulus as defined in any of the previous claims.
